(19) United States
(12) Patent Application Publication
(10) Pub. No.: US 2023/0357222 A1
(43) Pub. Date: Nov. 9, 2023

(54) SELECTIVE SMALL MOLECULE DEGRADERS OF CEREBLON

(71) Applicant: DANA-FARBER CANCER INSTITUTE, INC., Boston, MA (US)

(72) Inventors: Nathanael S. Gray, Boston, MA (US); Zhixiang He, Brookline, MA (US); Tinghu Zhang, Brookline, MA (US); Chelsea Powell, Brookline, MA (US); Eric S. Fischer, Chestnut Hill, MA (US); Jonathan Bushman, Boston, MA (US); Guangyan Du, Jamaica Plain, MA (US)

(73) Assignee: DANA-FARBER CANCER INSTITUTE, INC., Boston, MA (US)

(21) Appl. No.: 17/911,564

(22) PCT Filed: Mar. 16, 2021
(86) PCT No.: PCT/US21/22564

§ 371 Date: Sep. 14, 2022

Related U.S. Application Data
(60) Provisional application No. 62/990,670, filed on Mar. 17, 2020.

(51) Int. Cl. C07D 417/14; A61K 47/55

(52) U.S. Cl. CPC C07D 417/14; A61K 47/55

(57) ABSTRACT

Disclosed are bispecific compounds (degraders) that target cereblon (CRBN) for degradation. Also disclosed are pharmaceutical compositions containing the degraders and methods of using the compounds as research tools and to reduce adverse side effects associated with a cereblon targeted therapy.

### RELATED APPLICATIONS

This application claims the benefit of priority under 35 U.S.C. § 119(e) to U.S. Provisional Application No. 62/990,670, filed on Mar. 17, 2020, which is incorporated herein by reference in its entirety.

### GOVERNMENT LICENSE RIGHTS

This invention was made with government support under grant number R01 CA214608 awarded by the National Institutes of Health/National Cancer Institute and grant number 5 F31 CA210619-02 awarded by the National Institutes of Health. The government has certain rights in the invention.

### BACKGROUND OF THE INVENTION

An exciting emerging technology pertains to the use of selective small molecule degraders (also called PROTAC®s or degronimides) which are hetero-bifunctional small molecules that can induce degradation of a target protein by bringing it into proximity of an E3 ligase (Winter et al., Science 348(6241): 1376-81(20215)). When this ternary complex is formed, the E3 ligase ubiquitinates the target protein, leading to its proteasomal degradation. Two of the most commonly used E3 ligases for this strategy are cereblon (CRBN) and Von-Hippel-Lindau (VHL) tumor suppressor.

### SUMMARY OF THE INVENTION

The present invention discloses bispecific compounds that selectively target cereblon for degradation. A first aspect of the present invention is directed to a bispecific compound represented by formula (I):

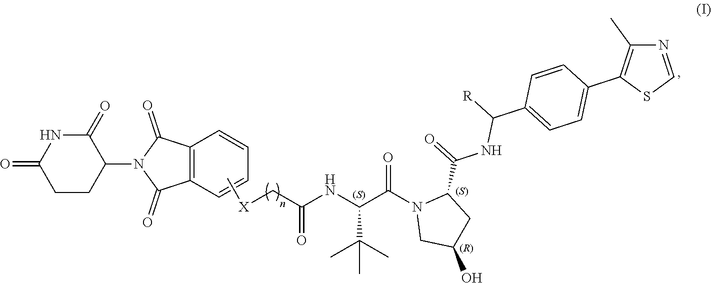

wherein
n represents an integer of 1-10, inclusive;
R represents H or methyl; and
X represents CH₂, N, or O;
or a pharmaceutically acceptable salt or stereoisomer thereof. The compounds include a targeting moiety that binds cereblon (CRBN) and a moiety that binds von Hippel-Lindau tumor suppressor (VHL) covalently attached to each other by an alkylene linker.

In some embodiments, n is 10.

In some embodiments, X is O.

Another aspect of the present invention is directed to a pharmaceutical composition that includes a therapeutically effective amount of the bispecific compound of formula (I) or a pharmaceutically acceptable salt or stereoisomer thereof, and a pharmaceutically acceptable carrier.

A further aspect of the present invention is directed to a method for making bispecific compounds of formula (I) or pharmaceutically acceptable salts or stereoisomers thereof.

In yet a further aspect, the present invention is directed to a method of reducing an adverse side effect associated with a cereblon targeted therapy, comprising administering to a patient in need thereof a therapeutically effective amount of the compound of formula (I) or pharmaceutically acceptable salt or stereoisomer thereof.

In yet a further aspect, the present invention is directed to a method of using a bispecific compound of formula (I) as a tool for studying CRBN biology via chemically induced knockdown. In some embodiments, the methods are conducted in vitro or in vivo in a non-human animal.

As shown in working examples, bispecific compounds of formula (I) showed high degree of selectivity and potency for CRBN, and induced CRBN degradation in the 10-nM range. Collectively, the present bispecific compounds may represent a set of new chemical tools for CRBN knockdown and may provide a potential modality for treating adverse side effects associated with a cereblon targeted therapy.

### BRIEF DESCRIPTION OF THE DRAWINGS

FIG. 1A is an immunoblot that shows the selective degradation of cereblon (CRBN) in MMl.s cells after 4-hour treatment with different concentrations (nM) of inventive bispecific compounds (Cpd) 1-3, pomalidomide (CRBN inhibitor) and DMSO (negative control).

FIG. 1B is an immunoblot that shows the selective degradation of cereblon (CRBN) in MM1.s cells after 4-hour treatment with different concentrations (nM) of compound 4, inventive bispecific compounds 5-6, pomalidomide and DMSO (negative control).

FIG. 2A is an immunoblot that shows the selective degradation of CRBN in MM1.s cells over a time course with 50-nM doses of inventive bispecific compounds 1-3 and DMSO (negative control).

FIG. 2B is an immunoblot that shows the selective degradation of CRBN in MM1.s cells over a time course with 50-nM doses of compound 4, inventive bispecific compounds 5-6 and DMSO (negative control).

FIG. 3A is a scatter plot depicting the changes in protein abundance in MM1.s cells after 6-hour treatment with pomalidomide (1 μM).

FIG. 3B is a scatter plot depicting the changes in protein abundance in MM1.s cells after 6-hour treatment with inventive bispecific compound 1 (50 nM).

FIG. 3C is a scatter plot depicting the changes in protein abundance in MM1.s cells after 6-hour treatment with inventive bispecific compound 6 (50 nM).

FIG. 3D is a scatter plot depicting the changes in protein abundance in HEK293T cells after 6-hour treatment with inventive bispecific compound 1 (50 nM).

FIG. 3E is a scatter plot depicting the changes in protein abundance in MOLT4 cells after 6-hour treatment with inventive bispecific compound 1 (50 nM).

FIG. 3F is a scatter plot depicting the changes in protein abundance in SK-N-DZ cells after 12-hour treatment with inventive bispecific compound 1 (50 nM).

FIG. 3G is a scatter plot depicting the changes in protein abundance in Kelly cells after 6-hour treatment with inventive bispecific compound 1 (50 nM).

FIG. 3H is a table illustrating the changes (log 2 fold change) in protein abundance in MM1.s cells after 6-hour treatment with inventive bispecific compounds 1 and 6 (50 nM) and pomalidomide (1 μM).

### DETAILED DESCRIPTION OF THE INVENTION

Unless defined otherwise, all technical and scientific terms used herein have the same meaning as is commonly understood by one of skill in the art to which the subject matter herein belongs. As used in the specification and the appended claims, unless specified to the contrary, the following terms have the meaning indicated in order to facilitate the understanding of the present invention.

As used in the description and the appended claims, the singular forms “a”, “an”, and “the” include plural referents unless the context clearly dictates otherwise. Thus, for example, reference to “a composition” includes mixtures of two or more such compositions, reference to “an inhibitor” includes mixtures of two or more such inhibitors, and the like.

Unless stated otherwise, the term “about” means within 10% (e.g., within 5%, 2% or 1%) of the particular value modified by the term “about.”

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

With respect to compounds of the present invention, and to the extent the following terms are used herein to further describe them, the following definitions apply.

As used herein, the term “alkyl” refers to a saturated linear or branched-chain monovalent hydrocarbon radical. In one embodiment, the alkyl radical is a C₁-C₁₈ group. In other embodiments, the alkyl radical is a C₀-C₆, C₀-C₅, C₀-C₃, C₁-C₁₂, C₁-C₈, C₁-C₆, C₁-C₅, C₁-C₄ or C₁-C₃ group (wherein C₀ alkyl refers to a bond). Examples of alkyl groups include methyl, ethyl, 1-propyl, 2-propyl, i-propyl, 1-butyl, 2-methyl-1-propyl, 2-butyl, 2-methyl-2-propyl, 1-pentyl, n-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, heptyl, octyl, nonyl, decyl, undecyl and dodecyl. In some embodiments, an alkyl group is a C₁-C₃ alkyl group. In some embodiments, an alkyl group is a C₁-C₂ alkyl group.

As used herein, the term “alkylene” refers to a straight or branched divalent hydrocarbon chain linking the rest of the molecule to a radical group, consisting solely of carbon and hydrogen, containing no unsaturation and having from one to 12 carbon atoms, for example, methylene, ethylene, propylene, n-butylene, and the like. The alkylene chain may be attached to the rest of the molecule through a single bond and to the radical group through a single bond. In some embodiments, the alkylene group contains one to 8 carbon atoms (C₁-C₈ alkylene). In other embodiments, an alkylene group contains one to 5 carbon atoms (C₁-C₅ alkylene). In other embodiments, an alkylene group contains one to 4 carbon atoms (C₁-C₄ alkylene). In other embodiments, an alkylene contains one to three carbon atoms (C₁-C₃ alkylene). In other embodiments, an alkylene group contains one to two carbon atoms (C₁-C₂ alkylene). In other embodiments, an alkylene group contains one carbon atom (C₁ alkylene).

Broadly, the bispecific compound comprises a targeting moiety that binds cereblon (CRBN) and a moiety that binds von Hippel-Lindau tumor suppressor (VHL) covalently attached to each other by an alkylene linker, wherein the compound has a structure represented by formula (I):

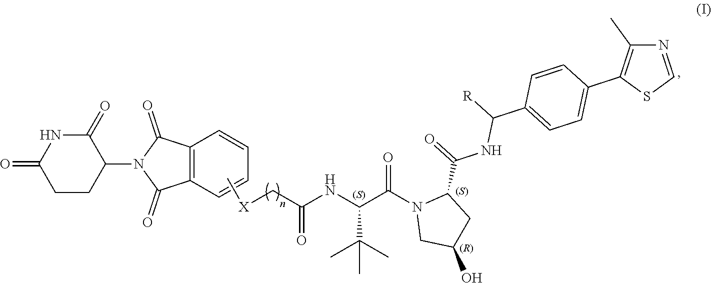

wherein
n represents an integer of 1-10, inclusive;
R represents H or methyl; and
X represents CH₂, N, or O;
or a pharmaceutically acceptable salt or stereoisomer thereof.

In some embodiments, n is 10.

In some embodiments, X is O.

### Linkers

The linker (“L”) provides a covalent attachment of the targeting moiety that binds CRBN to the degron. The structure of linker may not be critical, provided it does not substantially interfere with the activity of the targeting ligand or the degron.

The alkylene linkers are represented by formula (L1):

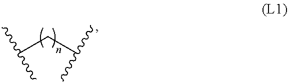

wherein n is an integer of 1-12 (“of” meaning inclusive), e.g., 1-12, 1-11, 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, 9-10 and 1, 2, 3, 4, 5, 6, 7, 8, 9 and 10 examples of which include:

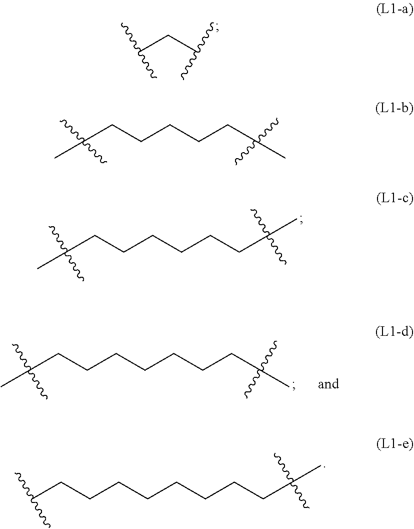

In some embodiments, the linker is represented by any one of structures:

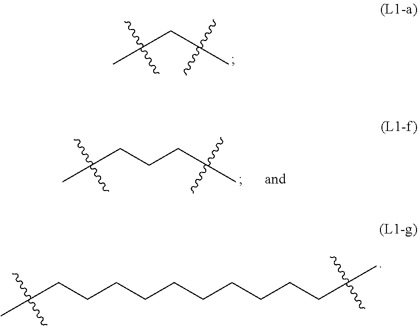

In some embodiments, the bispecific compounds of formula (I) have a structure represented by any one of formulae I-1 to 1-3:

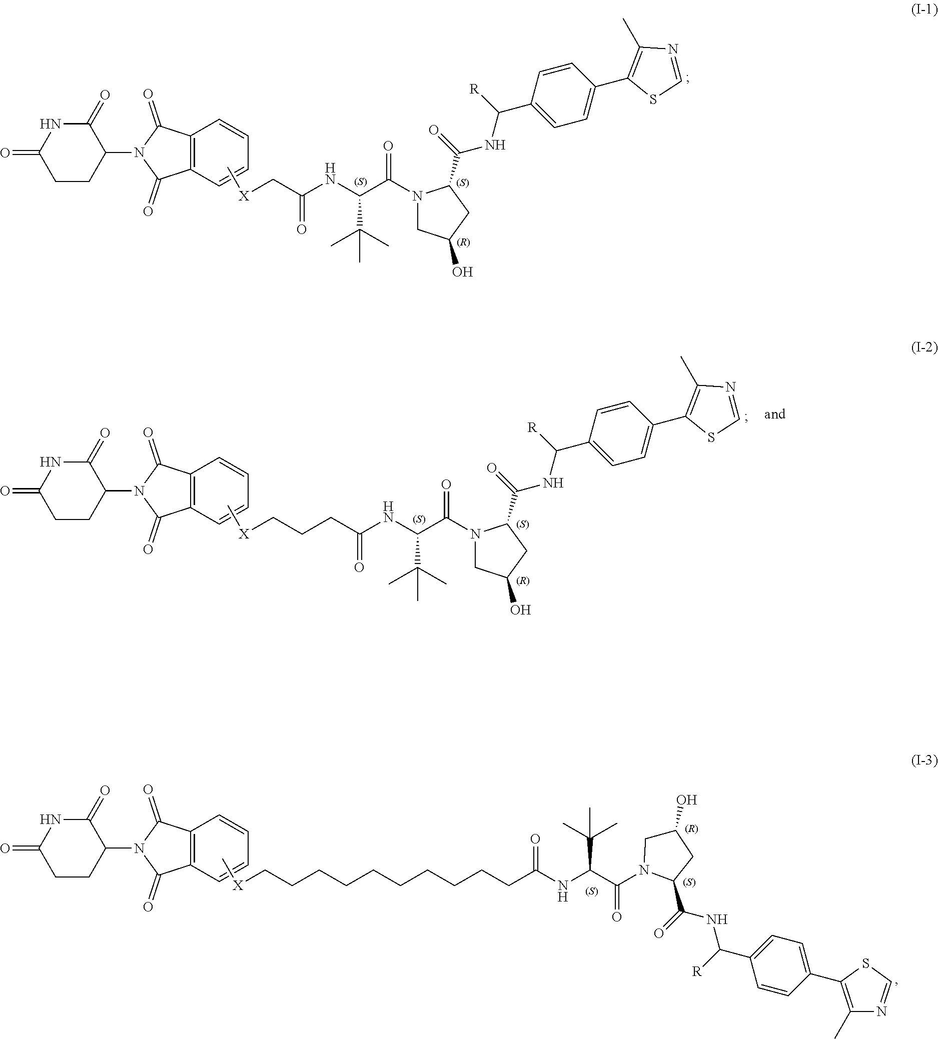

wherein
X represents CH₂, N, or O;
and R represents H or methyl;
or a pharmaceutically acceptable salt or stereoisomer thereof.

In some embodiments, the bispecific compound of formula (I) is represented by any one of structures 1-6:

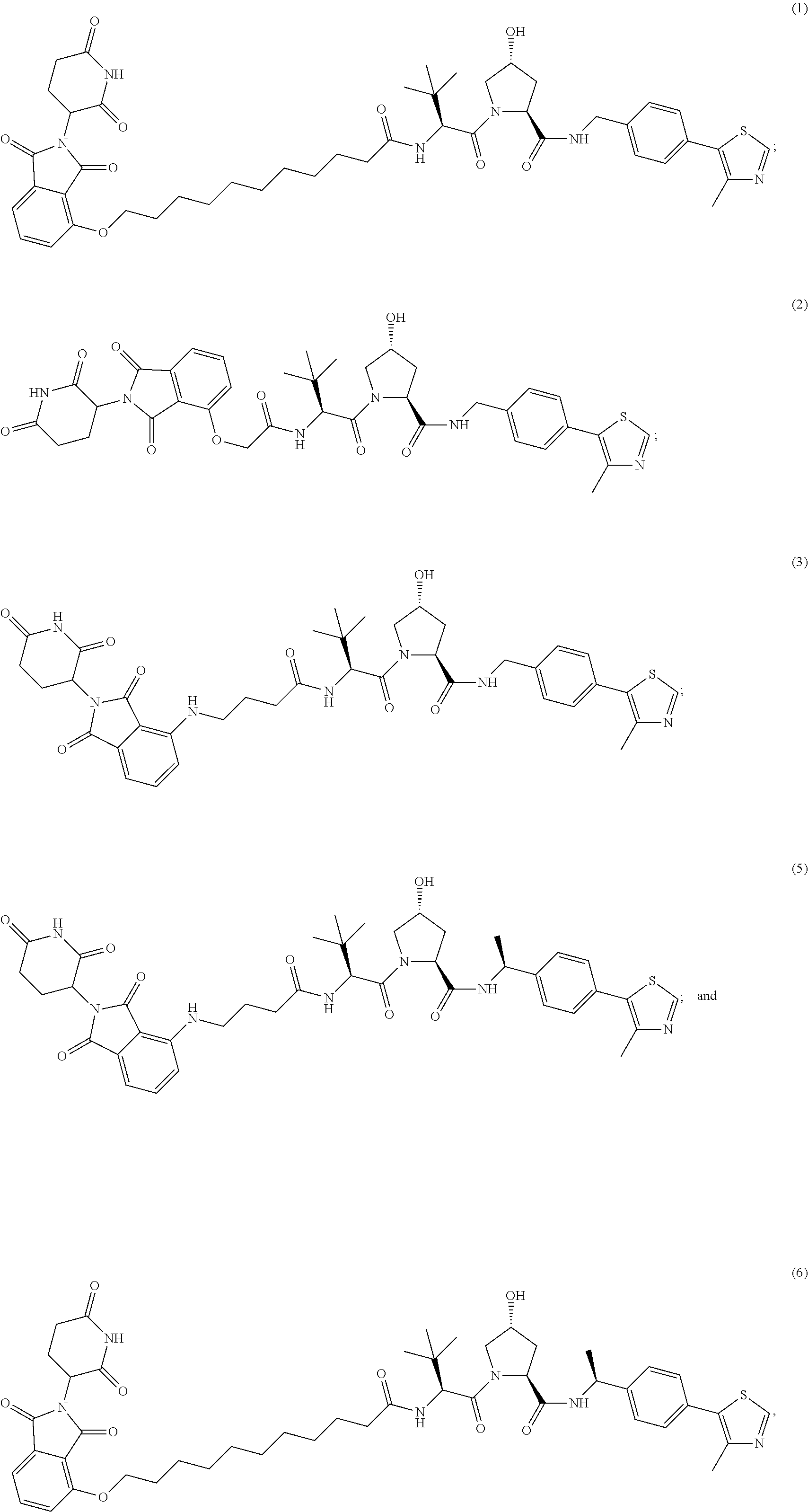

or a pharmaceutically acceptable salt and stereoisomer thereof.

Bispecific compounds of formula (I) may be in the form of a free acid or free base, or a pharmaceutically acceptable salt. As used herein, the term “pharmaceutically acceptable” in the context of a salt refers to a salt of the compound that does not abrogate the biological activity or properties of the compound, and is relatively non-toxic, i.e., the compound in salt form may be administered to a subject without causing undesirable biological effects (such as dizziness or gastric upset) or interacting in a deleterious manner with any of the other components of the composition in which it is contained. The term “pharmaceutically acceptable salt” refers to a product obtained by reaction of the compound of the present invention with a suitable acid or a base. Examples of pharmaceutically acceptable salts of the compounds of this invention include those derived from suitable inorganic bases such as Li, Na, K, Ca, Mg, Fe, Cu, Al, Zn and Mn salts. Examples of pharmaceutically acceptable, nontoxic acid addition salts are salts of an amino group formed with inorganic acids such as hydrochloride, hydrobromide, hydroiodide, nitrate, sulfate, bisulfate, phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, 4-methylbenzenesulfonate or p-toluenesulfonate salts and the like. Certain compounds of the invention can form pharmaceutically acceptable salts with various organic bases such as lysine, arginine, guanidine, diethanolamine or metformin.

In some embodiments, the bispecific compound of formula (I) is an isotopic derivative in that it has at least one desired isotopic substitution of an atom, at an amount above the natural abundance of the isotope, i.e., enriched. In one embodiment, the compound includes deuterium or multiple deuterium atoms. Substitution with heavier isotopes such as deuterium, i.e. 2H, may afford certain therapeutic advantages resulting from greater metabolic stability, for example, increased in vivo half-life or reduced dosage requirements, and thus may be advantageous in some circumstances.

As reflected in formula (I), bispecific compounds of formula (I) may have at least one chiral center and thus may be in the form of a stereoisomer, which as used herein, embraces all isomers of individual compounds that differ only in the orientation of their atoms in space. The term stereoisomer includes mirror image isomers (enantiomers which include the (R—) or (S—) configurations of the compounds), mixtures of mirror image isomers (physical mixtures of the enantiomers, and racemates or racemic mixtures) of compounds, geometric (cis/trans or E/Z, R/S) isomers of compounds and isomers of compounds with more than one chiral center that are not mirror images of one another (diastereoisomers). The chiral centers of the compounds may undergo epimerization in vivo; thus, for these compounds, administration of the compound in its (R—) form is considered equivalent to administration of the compound in its (S—) form. Accordingly, the compounds of the present invention may be made and used in the form of individual isomers and substantially free of other isomers, or in the form of a mixture of various isomers, e.g., racemic mixtures of stereoisomers.

In addition, bispecific compounds of formula (I) embrace N-oxides, crystalline forms (also known as polymorphs), active metabolites of the compounds having the same type of activity, tautomers, and unsolvated as well as solvated forms with pharmaceutically acceptable solvents such as water, ethanol, and the like, of the compounds. The solvated forms of the conjugates presented herein are also considered to be disclosed herein.

### Methods of Synthesis

In some embodiments, the present invention is directed to a method for making a bispecific compound of formula (I) or a pharmaceutically acceptable salt or stereoisomer thereof, of the invention. Broadly, the inventive bispecific compounds or pharmaceutically acceptable salts or stereoisomers thereof, may be prepared by any process known to be applicable to the preparation of chemically related compounds. The bispecific compounds of the present invention will be better understood in connection with the synthetic schemes that described in various working examples and which illustrate non-limiting methods by which the compounds of the invention may be prepared.

### Pharmaceutical Compositions

Another aspect of the present invention is directed to a pharmaceutical composition that includes a therapeutically effective amount of the bispecific compound of formula (I) or a pharmaceutically acceptable salt or stereoisomer thereof, and a pharmaceutically acceptable carrier. The term “pharmaceutically acceptable carrier,” as known in the art, refers to a pharmaceutically acceptable material, composition or vehicle, suitable for administering compounds of the present invention to mammals. Suitable carriers may include, for example, liquids (both aqueous and non-aqueous alike, and combinations thereof), solids, encapsulating materials, gases, and combinations thereof (e.g., semi-solids), and gases, that function to carry or transport the compound from one organ, or portion of the body, to another organ, or portion of the body. A carrier is “acceptable” in the sense of being physiologically inert to and compatible with the other ingredients of the formulation and not injurious to the subject or patient. Depending on the type of formulation, the composition may include one or more pharmaceutically acceptable excipients.

Broadly, bispecific compounds of formula (I) may be formulated into a given type of composition in accordance with conventional pharmaceutical practice such as conventional mixing, dissolving, granulating, dragée-making, levigating, emulsifying, encapsulating, entrapping and compression processes (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York). Parenteral (e.g., subcutaneous (s.c.), intravenous (i.v.), intramuscular (i.m.)) administration may be advantageous in that the bispecific compound may be administered relatively quickly such as in the case of a single-dose treatment and/or an acute condition. Other medically acceptable modes of administration may be selected within the sound discretion of the medical professional.

In some embodiments, the compositions are formulated for intravenous administration (e.g., systemic intravenous injection).

Accordingly, bispecific compounds of the present invention may be formulated into liquid compositions (e.g., solutions in which the compound is dissolved, suspensions in which solid particles of the compound are dispersed, emulsions, and solutions containing liposomes, micelles, or nanoparticles). Compounds may also be formulated for rapid, intermediate or extended release.

Injectable preparations may include sterile aqueous solutions or oleaginous suspensions. They may be formulated according to standard techniques using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. The effect of the compound may be prolonged by slowing its absorption, which may be accomplished by the use of a liquid suspension or crystalline or amorphous material with poor water solubility. Prolonged absorption of the compound from a parenterally administered formulation may also be accomplished by suspending the compound in an oily vehicle.

The bifunctional compounds may also be formulated for other types of administration, e.g., oral, in accordance with sound medical judgment.

### Dosage Amounts

As used herein, the term, “therapeutically effective amount” refers to an amount of a bispecific compound of formula (I) or a pharmaceutically acceptable salt or a stereoisomer thereof; or a composition including the bispecific compound of formula (I) or a pharmaceutically acceptable salt or a stereoisomer thereof, effective in producing the desired therapeutic response in a particular patient suffering from an adverse side effect associated with a cereblon targeted therapy, namely amelioration of the adverse side effect.

The total daily dosage of the bispecific compounds and usage thereof may be decided in accordance with standard medical practice, e.g., by the attending physician using sound medical judgment. The specific therapeutically effective dose for any particular subject may depend upon a variety of factors including the disease or disorder being treated and the severity thereof (e.g., its present status); the age, body weight, general health, sex and diet of the subject; the time of administration, route of administration, and rate of excretion of the specific compound employed; the duration of the treatment; drugs used in combination or coincidental with the bispecific compound; and like factors well known in the medical arts (see, for example, Goodman and Gilman's, “The Pharmacological Basis of Therapeutics”, 10th Edition, A. Gilman, J. Hardman and L. Limbird, eds., McGraw-Hill Press, 155-173, 2001).

Bispecific compounds of formula (I) may be effective over a wide dosage range. In some embodiments, the total daily dosage (e.g., for adult humans) may range from about 0.001 to about 1600 mg, from 0.01 to about 1600 mg, from 0.01 to about 500 mg, from about 0.01 to about 100 mg, from about 0.5 to about 100 mg, from 1 to about 100-400 mg per day, from about 1 to about 50 mg per day, and from about 5 to about 40 mg per day, and in yet other embodiments from about 10 to about 30 mg per day. Individual dosages may be formulated to contain the desired dosage amount depending upon the number of times the compound is administered per day. By way of example, capsules may be formulated with from about 1 to about 200 mg of compound (e.g., 1, 2, 2.5, 3, 4, 5, 10, 15, 20, 25, 50, 100, 150, and 200 mg). In some embodiments, individual dosages may be formulated to contain the desired dosage amount depending upon the number of times the compound is administered per day.

### Methods of Use

In some aspects, the present invention is directed to methods of reducing an adverse side effect associated with a cereblon targeted therapy, comprising administering to a patient in need thereof a therapeutically effective amount of the compound of formula (I) or pharmaceutically acceptable salt or stereoisomer thereof. Cereblon targeted therapies that might cause adverse side effects include PROteolysis TArgeting Chimera (PROTAC®) therapies that target CRBN and a target protein for degradation.

The methods of the present invention may entail administration of bispecific compounds of formula (I) or pharmaceutical compositions thereof to the patient in a single dose or in multiple doses (e.g., 1, 2, 3, 4, 5, 6, 7, 8, 10, 15, 20, or more doses). For example, the frequency of administration may range from once a day up to about once every eight weeks. In some embodiments, the frequency of administration ranges from about once a day for 1, 2, 3, 4, 5, or 6 weeks, and in other embodiments entails a 28-day cycle which includes daily administration for 3 weeks (21 days). In other embodiments, the bispecific compound may be dosed twice a day (BID) over the course of two and a half days (for a total of 5 doses) or once a day (QD) over the course of two days (for a total of 2 doses). In other embodiments, the bispecific compound may be dosed once a day (QD) over the course of five days.

In some aspects, the present invention is directed to methods of using bispecific compounds of formula (I) as a tool for studying CRBN biology via chemically induced knockdown.

In some embodiments, the methods are conducted in vitro. Cells may be human or non-human in nature. Representative cell lines include blood cancer cell lines such as dexamethasone sensitive human multiple myeloma cell, MMl.s, acute myeloid leukemia cells MOLM-13 and MOLT-4, human promyelocytic leukemia cell NB-4, and multiple myeloma cell OPM-2, as well as common “workhorse” cell lines like human embryonic kidney cell HEK293. The methods may also be conducted in vivo in a non-human animal such as a rodent (e.g., a mouse).

### Pharmaceutical Kits

The present compositions may be assembled into kits or pharmaceutical systems. Kits or pharmaceutical systems according to this aspect of the invention include a carrier or package such as a box, carton, tube or the like, having in close confinement therein one or more containers, such as vials, tubes, ampoules, or bottles, which contain the bispecific compound of formula (I) or a pharmaceutical composition thereof. The kits or pharmaceutical systems of the invention may also include printed instructions for using the compounds and compositions.

These and other aspects of the present invention will be further appreciated upon consideration of the following Examples, which are intended to illustrate certain particular embodiments of the invention but are not intended to limit its scope, as defined by the claims.

### EXAMPLES

### Example 1: Synthesis of (2S,4R)-1-((2S)-2-(11-((2-(2,6-dioxopiperidin-3-yl)-1,3-dioxoisoindolin-4-yl)oxy)undecanamido)-3,3-dimethylbutanoyl)-4-hydroxy-N-(4-(4-methylthiazol-5-yl)benzyl)pyrrolidine-2-carboxamide (1)

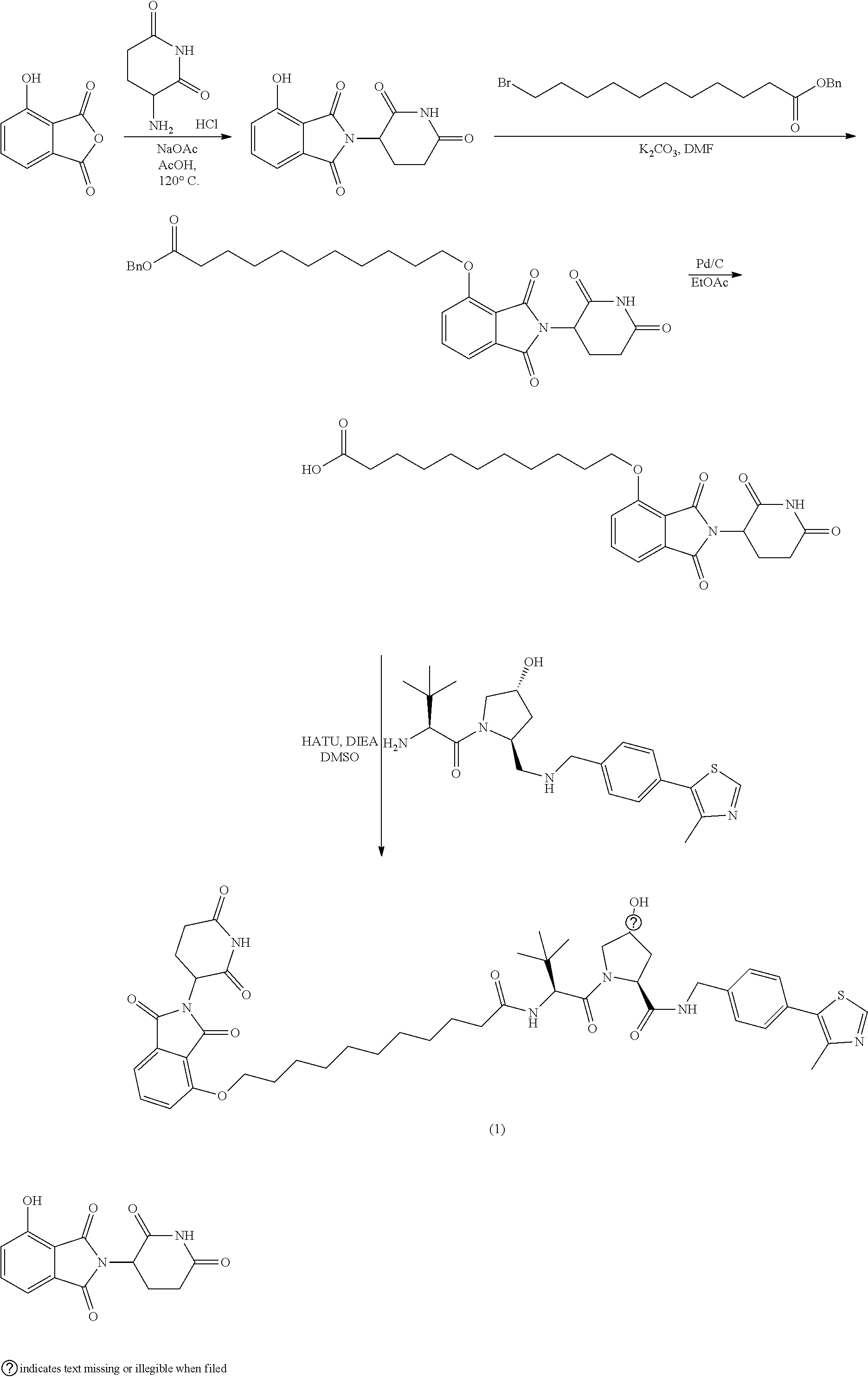

2-(2,6-Dioxopiperidin-3-yl)-4-hydroxyisoindoline-1,3-dione

To a solution of 4-hydroxyisobenzofuran-1,3-dione (1.64 g, 10 mmol) and 3-aminopiperidine-2,6-dione hydrochloride (1.65 g, 10 mmol) in acetic acid (30 mL) was added NaOAc (984 mg, 12 mmol), then the mixture solution was stirred at 120° C. for 12 h. After the reaction reached completion, the mixture was allowed to cool to room temperature and was filtered. The isolated solid was washed with water and hexane, and then air dried overnight to obtain the crude product (2.23 g, 82%) as a gray powder.

LCMS: 275 [M+H]⁺

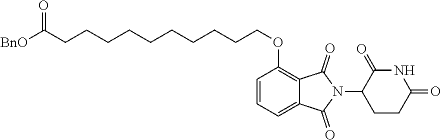 [SMILES: O=C1CCC(N2C(=O)c3cccc(OCCCCCCCCCCC(=O)OCc4ccccc4)c3C2=O)C(=O)N1]

Benzyl 11-((2-(2,6-dioxopiperidin-3-yl)-1,3-dioxoisoindolin-4-yl)oxy)undecanoate

To a solution of 2-(2,6-dioxopiperidin-3-yl)-4-hydroxyisoindoline-1,3-dione (200 mg, 0.73 mmol) and benzyl 11-bromoundecanoate (310 mg, 0.87 mmol) in DMF (4 mL) was added K₂CO₃ (152 mg, 1.1 mmol) and then the mixture was stirred at room temperature until the reaction reached completion. The mixture was filtered, and the filtrate was collected and then purified by high performance liquid chromatography (HPLC) (MeOH/water, 0.035% TFA) to obtain the product (180 mg, 37%) as a TFA salt.

LCMS: 549 [M+H]⁺.

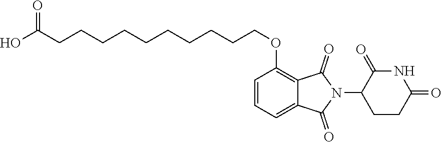 [SMILES: O=C(O)CCCCCCCCCCOc1cccc2c1C(=O)N(C1CCC(=O)NC1=O)C2=O]

11-((2-(2,6-Dioxopiperidin-3-yl)-1,3-dioxoisoindolin-4-yl)oxy)undecanoic acid

To a solution of benzyl 11-((2-(2,6-dioxopiperidin-3-yl)-1,3-dioxoisoindolin-4-yl)oxy)undecanoate (180 mg, 0.27 mmol) in EtOAc (20 mL) was added Pd/C (20 mg, 60% w.t. in mineral oil). The mixture was stirred under N₂ atmosphere until the reaction reached completion. The mixture was then filtered and the filtrate was concentrated in vacuo to obtain the product (105 mg, 82%), which was used in the next step without any purification.

LCMS: 459 [M+H]⁺.

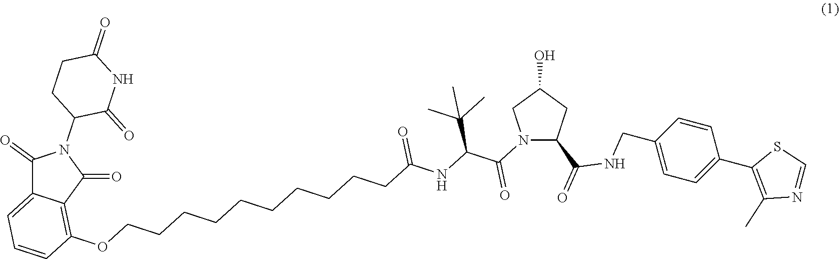 [SMILES: Cc1ncsc1-c1ccc(CNC(=O)[C@@H]2C[C@@H](O)CN2C(=O)[C@@H](NC(=O)CCCCCCCCCCOc2cccc3c2C(=O)N(C2CCC(=O)NC2=O)C3=O)C(C)(C)C)cc1]

To a solution of 11-((2-(2,6-dioxopiperidin-3-yl)-1,3-dioxoisoindolin-4-yl)oxy)undecanoic acid (26 mg, 0.037 mmol) and (4R)-3-methyl-L-valyl-4-hydroxy-N-[[4-(4-methyl-5-thiazolyl)phenyl]methyl]-L-prolinamide (26 mg, 0.037 mmol) in DMSO (1 mL) were added 1-[bis(dimethylamino)methylene]-1H-1,2,3-triazolo[4,5-b]pyridinium 3-oxide hexafluorophosphate (HATU) (17 mg, 0.044 mmol) and N,N-diisopropylethylamine (DIEA) (31 μL, 0.19 mmol). The mixture was stirred at room temperature for 1 h. The crude product was purified by HPLC (MeOH/water, 0.035% TFA) to obtain compound 1 (16 mg, 44%) as a TFA salt.

¹H NMR (500 MHz, DMSO-d6) δ 11.10 (s, 1H), 8.98 (s, 1H), 8.55 (t, J=6.1 Hz, 1H), 7.86-7.76 (m, 2H), 7.50 (d, J=8.6 Hz, 1H), 7.46-7.40 (m, 3H), 7.38 (d, J=8.4 Hz, 2H), 5.08 (dt, J=12.7, 7.2 Hz, 2H), 4.54 (d, J=9.3 Hz, 1H), 4.48-4.40 (m, 2H), 4.37-4.30 (m, 1H), 4.27-4.11 (m, 3H), 3.72-3.58 (m, 2H), 2.88 (ddd, J=16.9, 13.9, 5.5 Hz, 1H), 2.59 (dt, J=17.0, 2.7 Hz, 1H), 2.54 (s, 1H), 2.44 (s, 3H), 2.30-2.21 (m, 1H), 2.10 (ddd, J=14.2, 8.1, 6.2 Hz, 1H), 2.02 (dtt, J=11.8, 6.4, 3.4 Hz, 2H), 1.90 (ddd, J=12.9, 8.5, 4.6 Hz, 1H), 1.82-1.68 (m, 2H), 1.54-1.39 (m, 4H), 1.36-1.18 (m, 9H), 0.93 (s, 9H).

LCMS: 871 [M+H]⁺.

### Example 2: Synthesis of (2S,4R)-1-((2S)-2-(2-((2-(2,6-Dioxopiperidin-3-Yl)-1,3-Dioxoisoindolin-4-Yl)Oxy)Acetamido)-3,3-Dimethylbutanoyl)-4-Hydroxy-N-(4-(4-Methylthiazol-5-Yl)Benzyl)Pyrrolidine-2-Carboxamide (2)

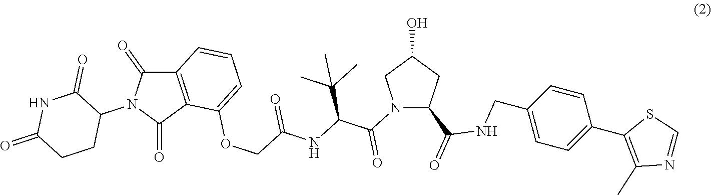 [SMILES: Cc1ncsc1-c1ccc(CNC(=O)[C@@H]2C[C@@H](O)CN2C(=O)[C@@H](NC(=O)COc2cccc3c2C(=O)N(C2CCC(=O)NC2=O)C3=O)C(C)(C)C)cc1]

Compound 2 was prepared in an analogous manner to compound 1 in Example 1 using benzyl 2-bromoacetate.

¹H NMR (500 MHz, DMSO-d6) δ 11.10 (s, 1H), 8.99 (s, 1H), 8.59 (d, J=5.1 Hz, 1H), 8.01 (dd, J=9.3, 3.6 Hz, 1H), 7.82 (t, J=7.9 Hz, 1H), 7.49 (d, J=7.2 Hz, 1H), 7.45 (d, J=8.6 Hz, 1H), 7.41 (d, J=2.9 Hz, 3H), 5.11 (dd, J=13.4, 5.3 Hz, 2H), 4.95 (dd, J=14.9, 2.8 Hz, 1H), 4.87 (dd, J=14.8, 2.2 Hz, 1H), 4.60 (dd, J=9.5, 3.5 Hz, 1H), 4.45 (dt, J=8.2, 4.2 Hz, 1H), 4.36 (d, J=5.5 Hz, 2H), 4.29 (ddd, J=15.7, 5.9, 2.9 Hz, 1H), 3.74-3.58 (m, 2H), 2.97-2.79 (m, 1H), 2.45 (s, 3H), 2.05 (dd, J=12.1, 5.6 Hz, 2H), 1.91 (ddd, J=12.9, 9.3, 4.5 Hz, 1H), 1.00 (s, 7H).

LCMS: 745 [M+H]⁺.

### Example 3: Synthesis of (2S,4R)-1-((2S)-2-(4-((2-(2,6-dioxopiperidin-3-yl)-1,3-dioxoisoindolin-4-yl)amino)butanamido)-3,3-dimethylbutanoyl)-4-hydroxy-N-(4-(4-methylthiazol-5-yl)benzyl)pyrrolidine-2-carboxamide (3)

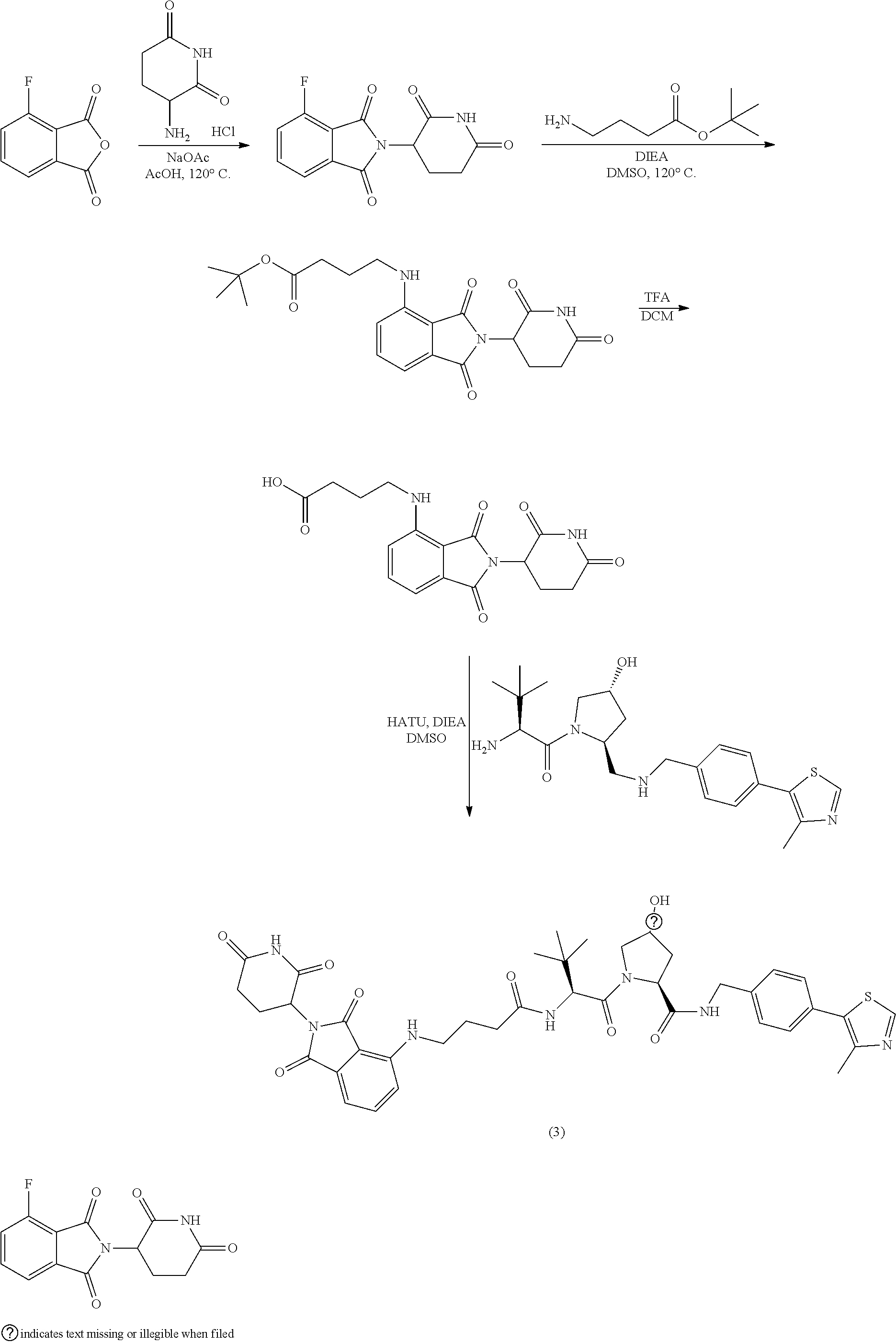

2-(2,6-Dioxopiperidin-3-yl)-4-fluoroisoindoline-1,3-dione

To a solution of 4-fluoroisobenzofuran-1,3-dione (1660 mg, 10 mmol) and 3-aminopiperidine-2,6-dione hydrochloride (1650 mg, 10 mmol) in acetic acid (30 mL) was added NaOAc (984 mg, 12 mmol), and the mixture solution was stirred at 120° C. for 12 h. After the reaction reached completion, the mixture was allowed to cool to room temperature and then was filtered. The isolated solid was washed with water and hexane and then was air dried overnight to obtain the crude product (1.68 g, 50%) as a gray powder.

LCMS: 277 [M+H]⁺

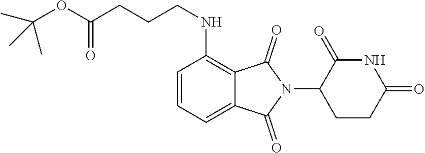 [SMILES: CC(C)(C)OC(=O)CCCNc1cccc2c1C(=O)N(C1CCC(=O)NC1=O)C2=O]

tert-Butyl 4-((2-(2,6-dioxopiperidin-3-yl)-1,3-dioxoisoindolin-4-yl)amino)butanoate

To a solution of 2-(2,6-dioxopiperidin-3-yl)-4-fluoroisoindoline-1,3-dione (55 mg, 0.2 mmol) and tert-butyl 4-aminobutanoate (32 mg, 0.2 mmol) in DMSO (2 mL) was added DIEA (66 μL, 0.4 mmol). The mixture was stirred at 120° C. for 1 h and then was purified by HPLC (MeOH/water, 0.035% TFA) to obtain the product as a TFA salt, which was used in the next step directly.

LCMS: 416 [M+H]⁺.

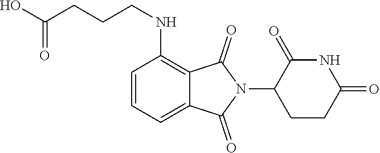 [SMILES: O=C(O)CCCNc1cccc2c1C(=O)N(C1CCC(=O)NC1=O)C2=O]

4-((2-(2,6-Dioxopiperidin-3-yl)-1,3-dioxoisoindolin-4-yl)amino)butanoic acid

To a solution of tert-butyl 4-((2-(2,6-dioxopiperidin-3-yl)-1,3-dioxoisoindolin-4-yl)amino)butanoate (0.2 mmol) in DCM (3 mL) was added TFA (1 mL). The mixture was stirred at room temperature for 1 h and then was concentrated in vacuo to obtain the product (34 mg, 36%) as a TFA salt, which was used in the next step without any purification.

LCMS: 360 [M+H]⁺.

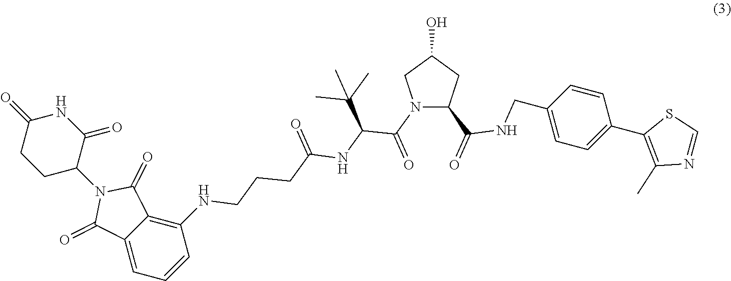 [SMILES: Cc1ncsc1-c1ccc(CNC(=O)[C@@H]2C[C@@H](O)CN2C(=O)[C@@H](NC(=O)CCCNc2cccc3c2C(=O)N(C2CCC(=O)NC2=O)C3=O)C(C)(C)C)cc1]

To a solution of 4-((2-(2,6-dioxopiperidin-3-yl)-1,3-dioxoisoindolin-4-yl)amino)butanoic acid (8 mg, 0.016 mmol) and (4R)-3-methyl-L-valyl-4-hydroxy-N-[[4-(4-methyl-5-thiazolyl)phenyl]methyl]-L-prolinamide (7 mg, 0.016 mmol) in DMSO (1 mL) were added HATU (7 mg, 0.019 mmol) and DIEA (8 μL, 0.048 mmol). The mixture was stirred at room temperature for 1 h and then was purified by HPLC (MeOH/water, 0.035% TFA) to obtain the product (3 mg, 21%) as a TFA salt.

¹H NMR (500 MHz, DMSO-d6) δ 11.09 (s, 1H), 9.00 (s, 1H), 8.56 (t, J=6.1 Hz, 1H), 7.99 (d, J=9.3 Hz, 1H), 7.59 (dd, J=8.6, 7.1 Hz, 1H), 7.43 (d, J=8.1 Hz, 2H), 7.39 (d, J=8.3 Hz, 2H), 7.11 (d, J=8.6 Hz, 1H), 7.02 (d, J=7.0 Hz, 1H), 6.64 (s, 1H), 5.06 (dd, J=12.7, 5.5 Hz, 1H), 4.57 (d, J=9.4 Hz, 1H), 4.47-4.34 (m, 4H), 4.22 (dd, J=15.9, 5.5 Hz, 1H), 3.68 (d, J=3.5 Hz, 2H), 2.89 (ddd, J=16.9, 13.7, 5.4 Hz, 1H), 2.66-2.57 (m, 1H), 2.45 (s, 3H), 2.36 (dd, J=14.6, 7.2 Hz, 1H), 2.26 (dt, J=14.4, 7.1 Hz, 1H), 2.08-1.99 (m, 2H), 1.91 (ddd, J=12.9, 8.6, 4.6 Hz, 1H), 1.79 (dq, J=17.9, 6.8 Hz, 2H), 0.95 (s, 9H).

LCMS: 772 [M+H]⁺.

### Example 4: Synthesis of (2S,4R)-1-4175)-17-(Tert-Butyl)-1-((2-(2,6-Dioxopiperidin-3-Yl)-1,3-Dioxoisoindolin-4-Yl)Amino)-15-Oxo-3,6,9,12-Tetraoxa-16-Azaoctadecan-18-Oyl)-4-Hydroxy-N-(4-(4-Methylthiazol-5-Yl)Benzyl)Pyrrolidine-2-Carboxamide (4)

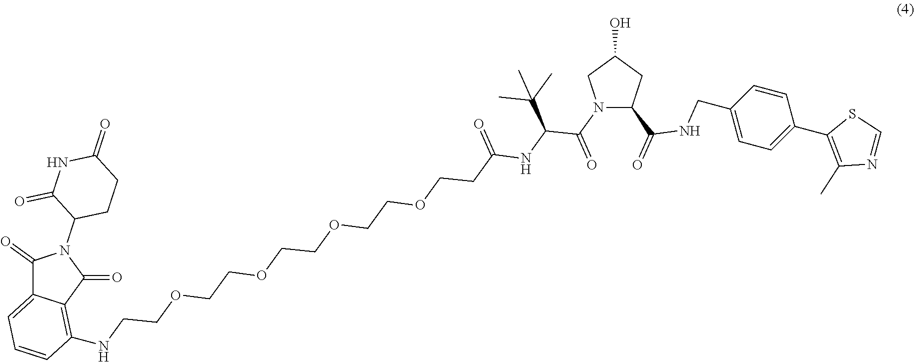 [SMILES: Cc1ncsc1-c1ccc(CNC(=O)[C@@H]2C[C@@H](O)CN2C(=O)[C@@H](NC(=O)CCOCCOCCOCCOCCNc2cccc3c2C(=O)N(C2CCC(=O)NC2=O)C3=O)C(C)(C)C)cc1]

Compound 4 was prepared in an analogous manner to compound 3 in Example 3 using 3,6,9,12-tetraoxapentadecan-15-oic acid (12 mg, 29%).

¹H NMR (500 MHz, DMSO-d6) δ 11.09 (s, 1H), 8.99 (s, 1H), 8.56 (t, J=6.1 Hz, 1H), 7.90 (d, J=9.4 Hz, 1H), 7.58 (dd, J=8.6, 7.0 Hz, 1H), 7.49-7.32 (m, 4H), 7.14 (d, J=8.6 Hz, 1H), 7.04 (d, J=7.1 Hz, 1H), 6.60 (s, 1H), 5.05 (dd, J=12.7, 5.5 Hz, 1H), 4.55 (d, J=9.4 Hz, 1H), 4.47-4.39 (m, 2H), 4.35 (dp, J=4.5, 2.4 Hz, 1H), 4.22 (dd, J=15.9, 5.5 Hz, 1H), 3.75-3.38 (m, 21H), 2.88 (ddd, J=16.8, 13.7, 5.4 Hz, 1H), 2.63-2.55 (m, 1H), 2.55-2.53 (m, 1H), 2.44 (s, 3H), 2.34 (dt, J=14.7, 6.2 Hz, 1H), 2.08-1.98 (m, 2H), 1.90 (ddd, J=12.9, 8.6, 4.6 Hz, 1H), 0.93 (s, 9H).

LCMS: 934 [M+H]⁺.

### Example 5: Synthesis of (2S,4R)-1-((25)-2-(4-((2-(2,6-Dioxopiperidin-3-Yl)-1,3-Dioxoisoindolin-4-Yl)Amino)Butanamido)-3,3-Dimethylbutanoyl)-4-Hydroxy-N-((S)-1-(4-(4-Methylthiazol-5-Yl)Phenyl)Ethyl)Pyrrolidine-2-Carboxamide (5)

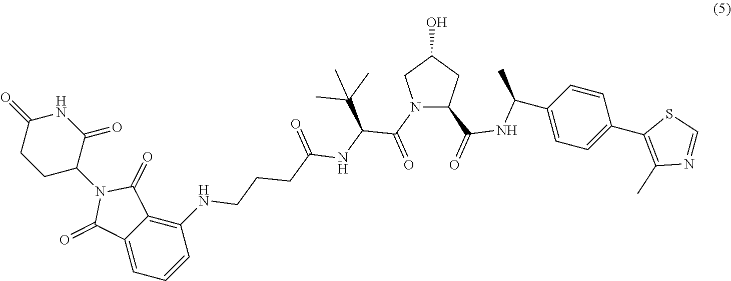 [SMILES: Cc1ncsc1-c1ccc([C@H](C)NC(=O)[C@@H]2C[C@@H](O)CN2C(=O)[C@@H](NC(=O)CCCNc2cccc3c2C(=O)N(C2CCC(=O)NC2=O)C3=O)C(C)(C)C)cc1]

Compound 5 was prepared in an analogous manner to compound 3 in Example 3 using (2S,4R)-1-((S)-2-amino-3,3-dimethylbutanoyl)-4-hydroxy-N-((S)-1-(4-(4-methylthiazol-5-yl) phenyl)ethyl)pyrrolidine-2-carboxamide dihydrochloride (20.2 mg, 61%).

¹H NMR (500 MHz, DMSO-d6) δ 11.10 (s, 1H), 9.00 (s, 1H), 8.37 (d, J=7.8 Hz, 1H), 7.94 (d, J=9.2 Hz, 1H), 7.59 (dd, J=8.6, 7.1 Hz, 1H), 7.44 (d, J=8.1 Hz, 2H), 7.39 (d, J=8.0 Hz, 2H), 7.11 (d, J=8.7 Hz, 1H), 7.03 (d, J=7.0 Hz, 1H), 6.65 (s, 1H), 5.06 (dd, J=12.7, 5.4 Hz, 1H), 4.92 (p, J=7.1 Hz, 1H), 4.55 (d, J=9.3 Hz, 1H), 4.43 (t, J=8.0 Hz, 1H), 4.29 (t, J=3.8 Hz, 1H), 3.63 (d, J=3.2 Hz, 2H), 2.89 (ddd, J=16.7, 13.7, 5.4 Hz, 1H), 2.65-2.57 (m, 2H), 2.46 (s, 3H), 2.34 (dt, J=14.7, 7.4 Hz, 1H), 2.25 (dt, J=14.6, 7.2 Hz, 1H), 2.03 (dtd, J=12.9, 7.9, 7.1, 3.5 Hz, 2H), 1.85-1.72 (m, 3H), 0.95 (s, 9H).

LCMS: 786 [M+H]⁺.

### Example 6: Synthesis of (2S,4R)-1-((2S)-2-(11-((2-(2,6-Dioxopiperidin-3-Yl)-1,3-Dioxoisoindolin-4-Yl)Oxy)Undecanamido)-3,3-Dimethylbutanoyl)-4-Hydroxy-N-((S)-1-(4-(4-Methylthiazol-5-Yl)Phenyl)Ethyl)Pyrrolidine-2-Carboxamide (6)

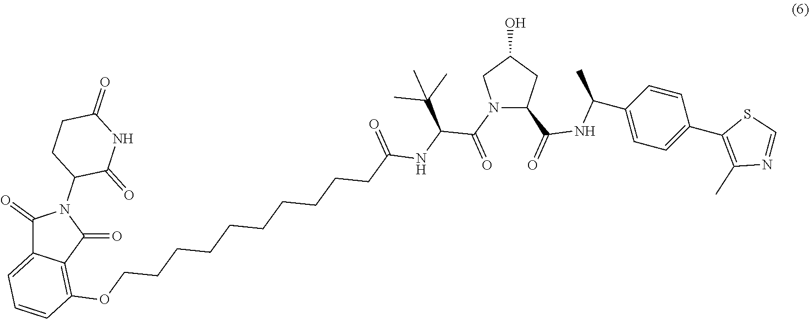 [SMILES: Cc1ncsc1-c1ccc([C@H](C)NC(=O)[C@@H]2C[C@@H](O)CN2C(=O)[C@@H](NC(=O)CCCCCCCCCCOc2cccc3c2C(=O)N(C2CCC(=O)NC2=O)C3=O)C(C)(C)C)cc1]

Compound 6 was prepared in an analogous manner to compound 1 in Example 1 using (2S,4R)-1-((S)-2-amino-3,3-dimethylbutanoyl)-4-hydroxy-N-((S)-1-(4-(4-methylthiazol-5-yl) phenyl)ethyl)pyrrolidine-2-carboxamide dihydrochloride (14.2 mg, 65%).

¹H NMR (500 MHz, DMSO-d6) δ 11.10 (s, 1H), 9.00 (s, 1H), 8.37 (d, J=7.8 Hz, 1H), 7.86-7.76 (m, 2H), 7.52 (d, J=8.6 Hz, 1H), 7.46-7.41 (m, 3H), 7.39 (d, J=8.1 Hz, 2H), 5.09 (dd, J=12.8, 5.4 Hz, 1H), 4.92 (p, J=7.2 Hz, 1H), 4.52 (d, J=9.3 Hz, 1H), 4.43 (t, J=8.0 Hz, 1H), 4.28 (t, J=3.6 Hz, 1H), 4.21 (t, J=6.4 Hz, 2H), 3.67-3.55 (m, 2H), 2.89 (ddd, J=16.9, 13.8, 5.4 Hz, 1H), 2.61 (d, J=3.4 Hz, 2H), 2.46 (s, 3H), 2.26 (dd, J=14.4, 7.5 Hz, 1H), 2.17-2.09 (m, 1H), 2.08-1.98 (m, 2H), 1.76 (p, J=6.8, 6.1 Hz, 2H), 1.47-1.23 (m, 15H), 0.94 (s, 9H).

LCMS: 885 [M+H]⁺.

### Example 7: Cereblon (CRBN) Degradation in MMl.s Cells with Bispecific Compounds 1-6

### Cell Culture

MM1.S cells were generously provided by James Bradner (Dana-Farber Cancer Institute (DFCI), Boston, MA). MM1.S cells were cultured in Roswell Park Memorial Institute (RPMI)-1640 media containing L-glutamine, supplemented with 10% fetal bovine serum (FBS) and 1% penicillin/streptomycin. Mycoplasma testing was performed on a monthly basis and all lines were negative.

### Cell Viability Assays

Cell viability was evaluated using the CellTiter-Glo® Luminescent Cell Viability Assay (Promega™) following the manufacturer's standards.

### Immunoblotting

Cells were washed with ice cold phosphate-buffered saline (PBS) before being lysed with Cell Lysis Buffer (Cell Signaling Technology®) supplemented with protease and phosphatase inhibitor cocktails (Roche®) at 4° C. for 15 minutes. The cell lysate was vortexed before being centrifuged at 14,000×g for 20 min at 4° C. Protein in cell lysate was quantified by bicinchoninic acid (BCA) assay (Pierce™). Primary antibodies used in this study include CDK9 (Cell Signaling Technology®, 2316S), CKlα (Abcam®, ab206652), CRBN (Novus Biologicals®, NBP1-91810), eRF3/GSPT1 (Abcam®, ab49878), IKZF1 (Ikaros) (Cell Signaling Technology®, 5443S), IKZF3 (Aiolos) (Cell Signaling Technology®, 15103S), VHL (Cell Signaling Technology®, 68547S), and vinculin (Abcam®, ab130007). Blot quantification was performed using Image Studio 4.0 software, normalizing to loading controls.

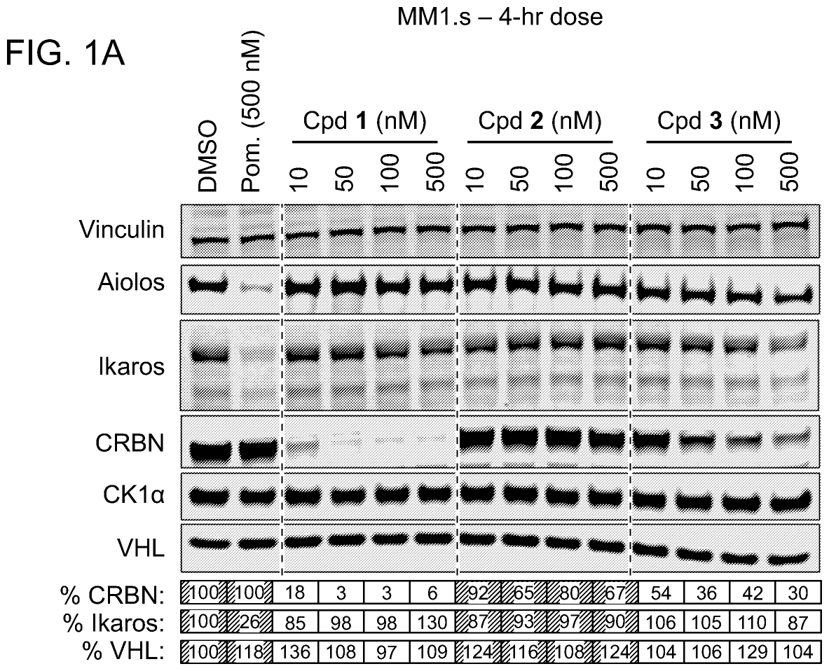

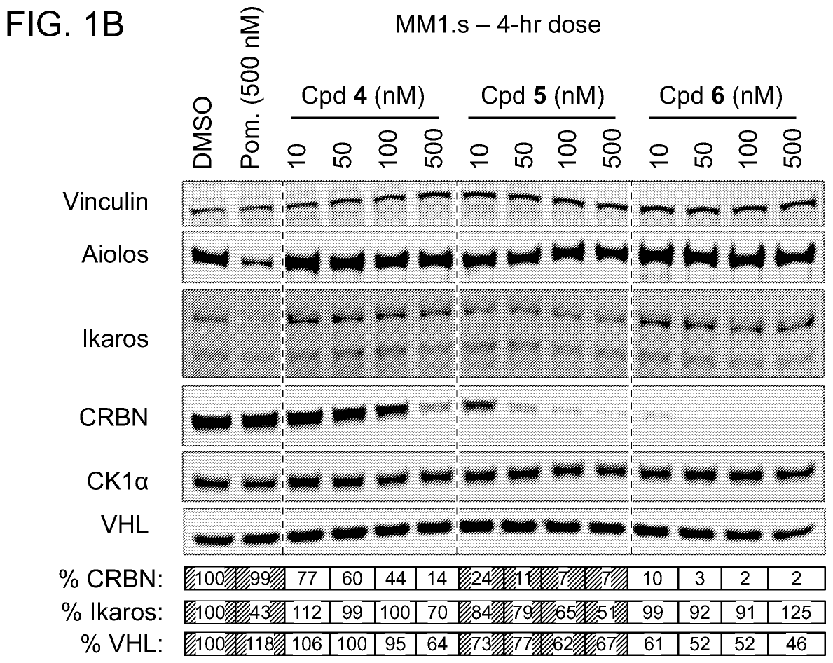

The results illustrated in FIG. 1A and FIG. 1B show that 4-h dose titration treatments in MMl.s cells indicated that bispecific compounds 1 and 6 were highly potent and selective degraders of CRBN with virtually complete degradation of CRBN being seen around 50 nM.

### Example 8: Cereblon (CRBN) Degradation in MMl.s Cells with Bispecific Compounds 1-6 in Time Course Experiment

The experimental protocol is set forth above in Example 7.

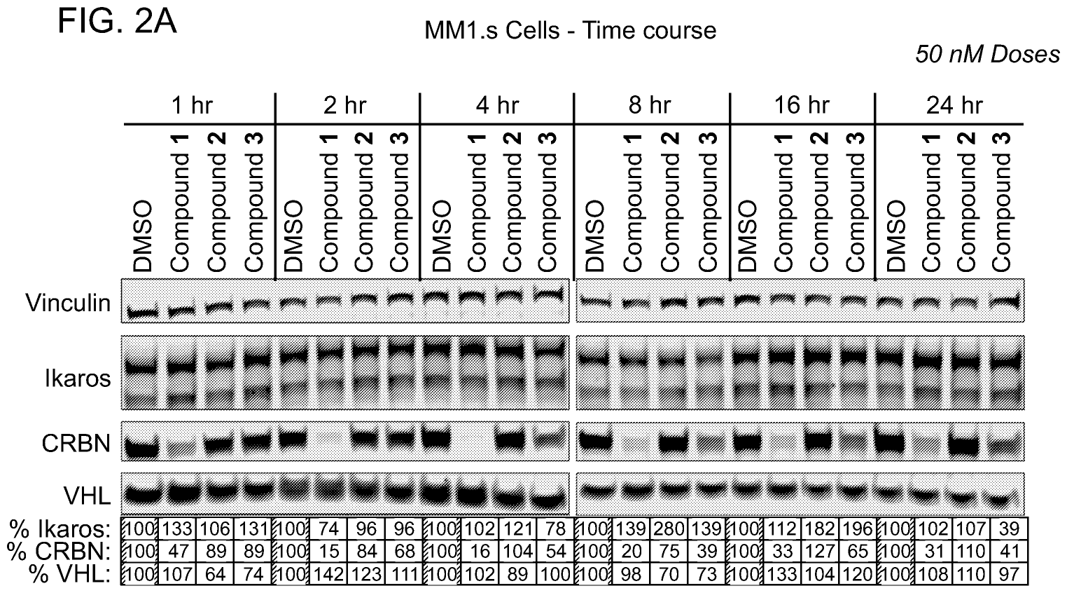

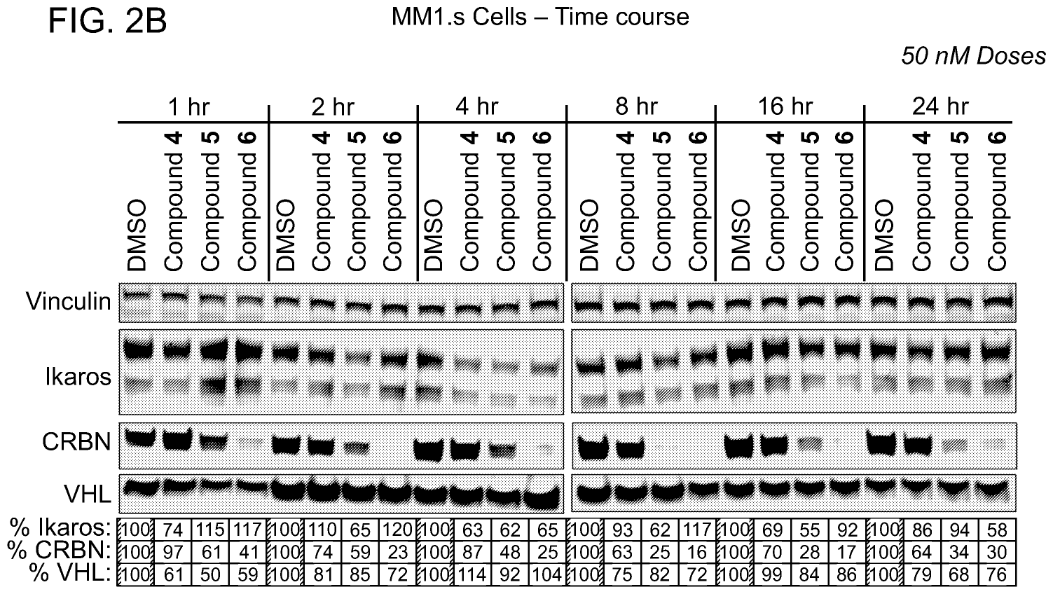

The results illustrated in FIG. 2A and FIG. 2B show that CRBN degradation from bispecific compounds 1 and 6 could be seen as early as 1 h after treatment, with peak degradation occurring between 2-16 h.

### Example 9: Proteomic Analysis

Sample preparation TMT LC-MS3 mass spectrometry

MM1.s cells were treated with DMSO or 50 nM of bispecific compound 1 or 6 in biological triplicates for 6 hours and cells harvested by centrifugation. Lysis buffer (8 M Urea, 50 mM NaCl, 50 mM 4-(2hydroxyethyl)-1-piperazineethanesulfonic acid (EPPS) pH 8.5, Protease and Phosphatase inhibitors from Roche®) was added to the cell pellets and homogenized by 20 passes through a 21 gauge (1.25 in. long) needle to achieve a cell lysate with a protein concentration between 1-4 mg mL-1. A micro-BCA assay (Pierce™) was used to determine the final protein concentration in the cell lysate. 200 μg of protein for each sample were reduced and alkylated as described in Donovan et al., Elife 7:e38430 (2018).

Proteins were precipitated using methanol/chloroform. In brief, four volumes of methanol were added to the cell lysate, followed by one volume of chloroform, and finally three volumes of water. The mixture was vortexed and centrifuged to separate the chloroform phase from the aqueous phase. The precipitated protein was washed with three volumes of methanol, centrifuged and the resulting washed precipitated protein was allowed to air dry. Precipitated protein was resuspended in 4 M Urea, 50 mM HEPES pH 7.4, followed by dilution to 1 M urea with the addition of 200 mM EPPS, pH 8. Proteins were first digested with LysC (1:50; enzyme:protein) for 12 hours at room temperature. The LysC digestion was diluted to 0.5 M Urea with 200 mM EPPS pH 8 followed by digestion with trypsin (1:50; enzyme:protein) for 6 hours at 37° C. Tandem mass tag (TMT) reagents (Thermo Fisher Scientific) were dissolved in anhydrous acetonitrile (ACN) according to manufacturer's instructions.

Anhydrous ACN was added to each peptide sample to a final concentration of 30% v/v, and labeling was induced with the addition of TMT reagent to each sample at a ratio of 1:4 peptide:TMT label. The 10-plex labeling reactions were performed for 1.5 hours at room temperature and the reaction quenched by the addition of hydroxylamine to a final concentration of 0.3% for 15 minutes at room temperature. The sample channels were combined at a 1:1:1:1:1:1:1:1:1:1 ratio, desalted using C18 solid phase extraction cartridges (Waters®) and analyzed by LC-MS for channel ratio comparison. Samples were then combined using the adjusted volumes determined in the channel ratio analysis and dried down in a speed vacuum. The combined sample was then resuspended in 1% formic acid, and acidified (pH 2-3) before being subjected to desalting with C18 SPE (Sep-Pak®, Waters®). Samples were then offline fractionated into 96 fractions by high pH reverse-phase HPLC (Agilent® LC1260) through an Aeris peptide Xb-C18 column (Phenomenex®) with mobile phase A containing 5% acetonitrile and 10 mM NH₄HCO₃ in LC-MS grade H₂O, and mobile phase B containing 90% acetonitrile and 10 mM NH₄HCO₃ in LC-MS grade H₂O (both pH 8.0). The 96 resulting fractions were then pooled in a non-continuous manner into 24 fractions and these fractions were used for subsequent mass spectrometry analysis.

Data were collected using an Orbitrap Fusion™ Lumos™ mass spectrometer (Thermo Fisher Scientific, San Jose, CA, USA) coupled with a Proxeon EASY-nLC™ 1200 LC pump (Thermo Fisher Scientific). Peptides were separated on an EasySpray™ ES803 75 μm inner diameter microcapillary column (ThermoFisher Scientific). Peptides were separated using a 190 min gradient of 6-27% acetonitrile in 1.0% formic acid with a flow rate of 350 nL/min.

Each analysis used an MS3-based TMT method as described in McAlister et al., Anal. Chem. 86(14):7150-7158 (2014). The data were acquired using a mass range of m/z 340-1350, resolution 120,000, automatic gain control (AGC) target 0.5×106, maximum injection time 100 ms, dynamic exclusion of 120 seconds for the peptide measurements in the Orbitrap. Data dependent MS2 spectra were acquired in the ion trap with a normalized collision energy (NCE) set at 35%, AGC target set to 1.8×104 and a maximum injection time of 120 ms. MS3 scans were acquired in the Orbitrap with a higher energy collision dissociation (HCD) set to 55%, AGC target set to 2×105, maximum injection time of 150 ms, resolution at 50,000 and with a maximum synchronous precursor selection (SPS) precursors set to 10.

LC-MS data analysis

Proteome Discoverer 2.2 (Thermo Fisher Scientific) was used for .RAW file processing and controlling peptide and protein level false discovery rates, assembling proteins from peptides, and protein quantification from peptides. MS/MS spectra were searched against a Uniprot human database (September 2016) with both the forward and reverse sequences. Database search criteria are as follows: tryptic with two missed cleavages, a precursor mass tolerance of 10 ppm, fragment ion mass tolerance of 0.6 Da, static alkylation of cysteine (57.02146 Da), static TMT labelling of lysine residues and N-termini of peptides (229.16293 Da), and variable oxidation of methionine (15.99491 Da). TMT reporter ion intensities were measured using a 0.003 Da window around the theoretical m/z for each reporter ion in the MS3 scan. Peptide spectral matches with poor quality MS3 spectra were excluded from quantitation (summed signal-to-noise across 10 channels<100 and precursor isolation specificity<0.5), and resulting data was filtered to only include proteins that had a minimum of 2 unique peptides identified. Reporter ion intensities were normalized and scaled using in-house scripts in the R framework (R Development Core Team (2008). R: A language and environment for statistical computing. R Foundation for Statistical Computing, Vienna, Austria). Statistical analysis was carried out using the limma package within the R framework (Ritchie et al., Nucleic Acids Res. 43:e74 (2015).

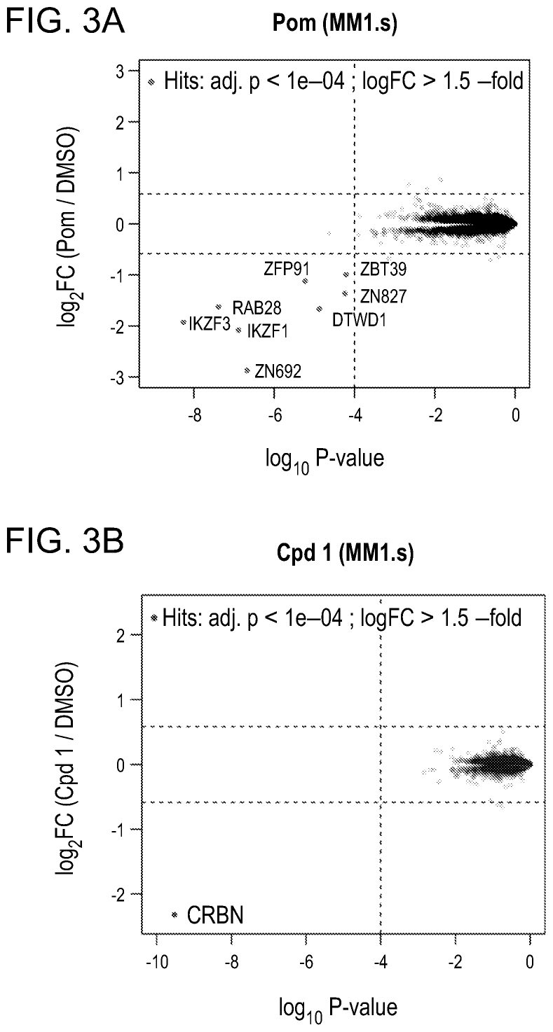

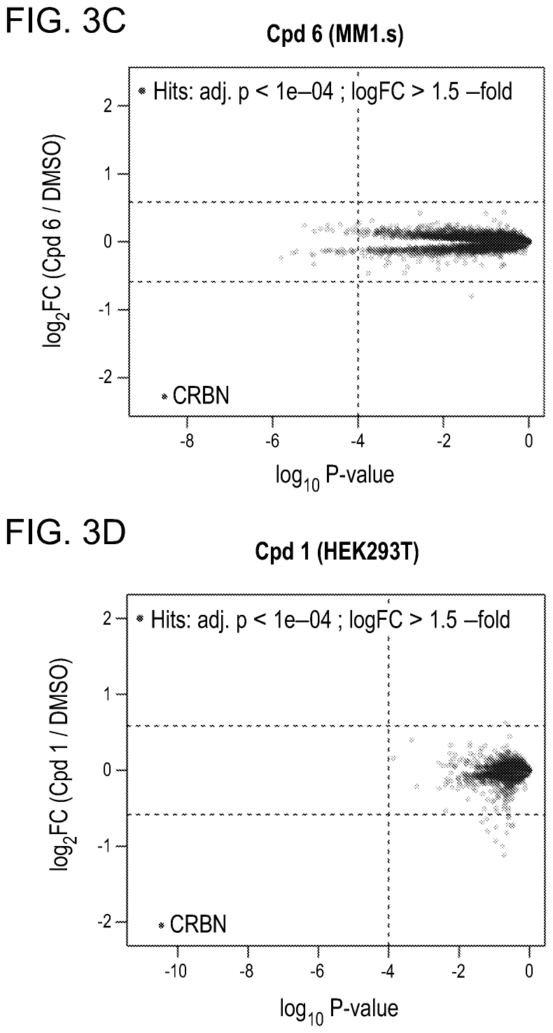

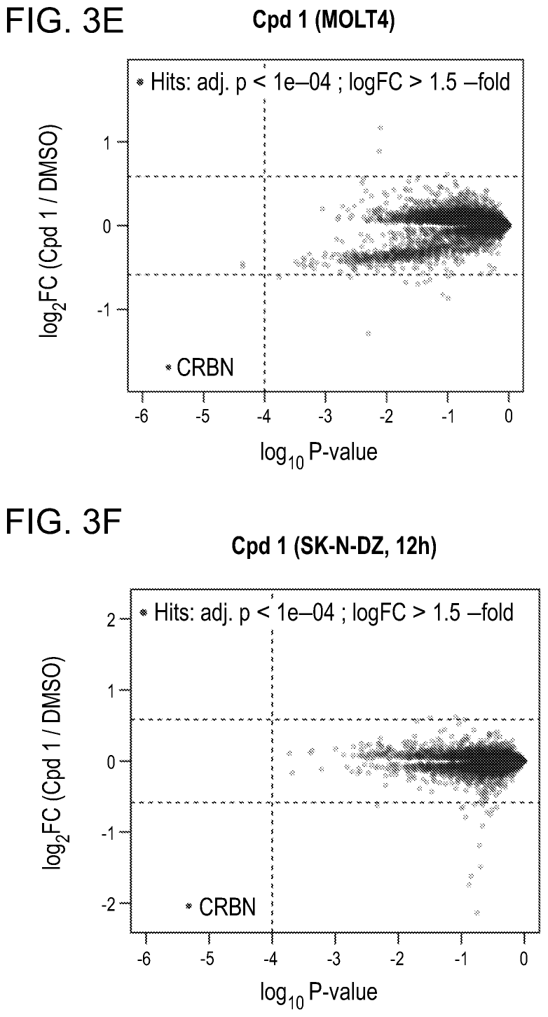

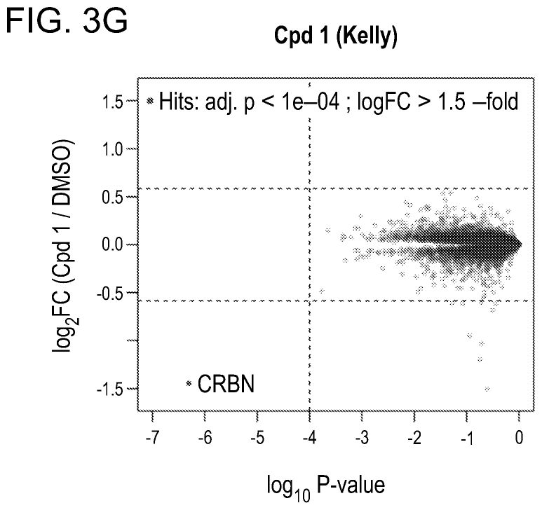

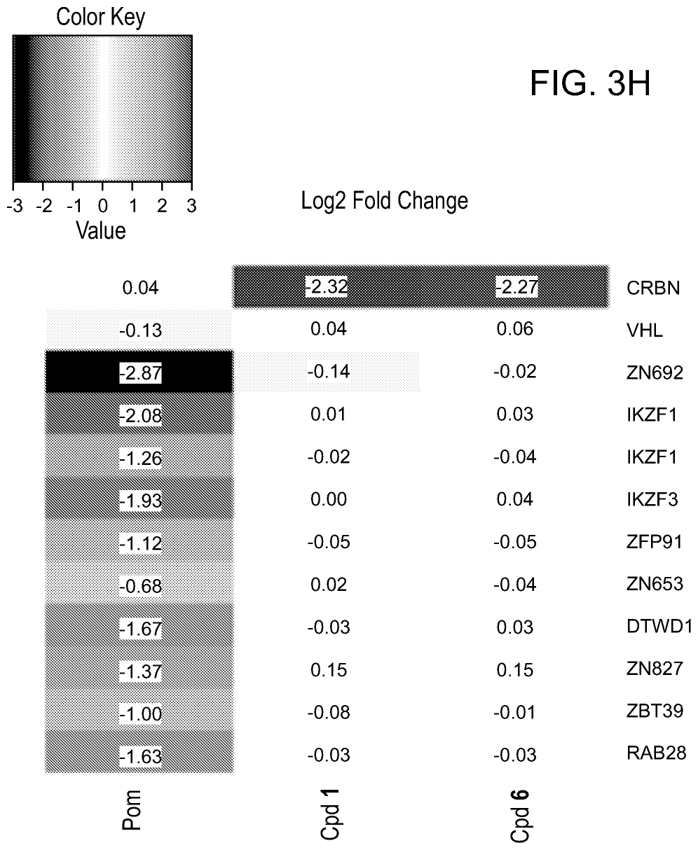

The results illustrated in FIG. 3A to FIG. 3C and FIG. 3H show that only CRBN was significantly degraded in MM1.s cells by inventive bispecific compounds 1 and 6 by proteomic analysis, validating the high degree of selectivity seen by western blotting. Similar results were observed in HEK293T, MOLT4, SK-N-DZ and Kelly cells with inventive bispecific compound 1 (FIG. 3D-FIG. 3G).

All patent publications and non-patent publications are indicative of the level of skill of those skilled in the art to which this invention pertains. All these publications are herein incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A bispecific compound, wherein the bispecific compound has a structure represented by formula (I): 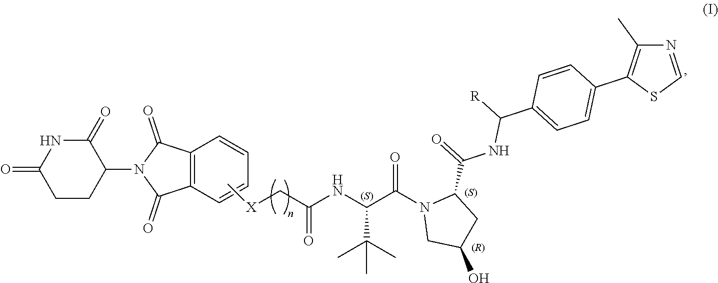
wherein
n represents an integer of 1-10, inclusive;
R represents H or methyl; and
X represents CH₂, N, or O;
or a pharmaceutically acceptable salt or stereoisomer thereof.

2. The bispecific compound of claim 1, wherein n is 10.

3. The bispecific compound of claim 1, wherein X is O.

4. The bispecific compound of claim 1, wherein the linker has a structure selected from the group consisting of:

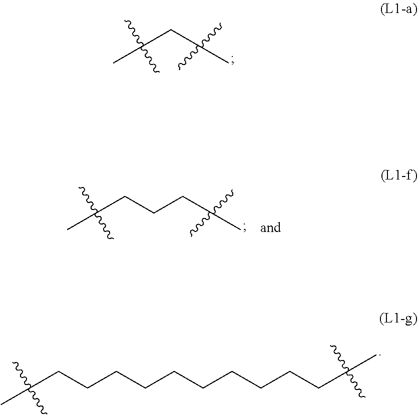

5. The bispecific compound of claim 1, which is represented by any one of structures I-1 to I 3: 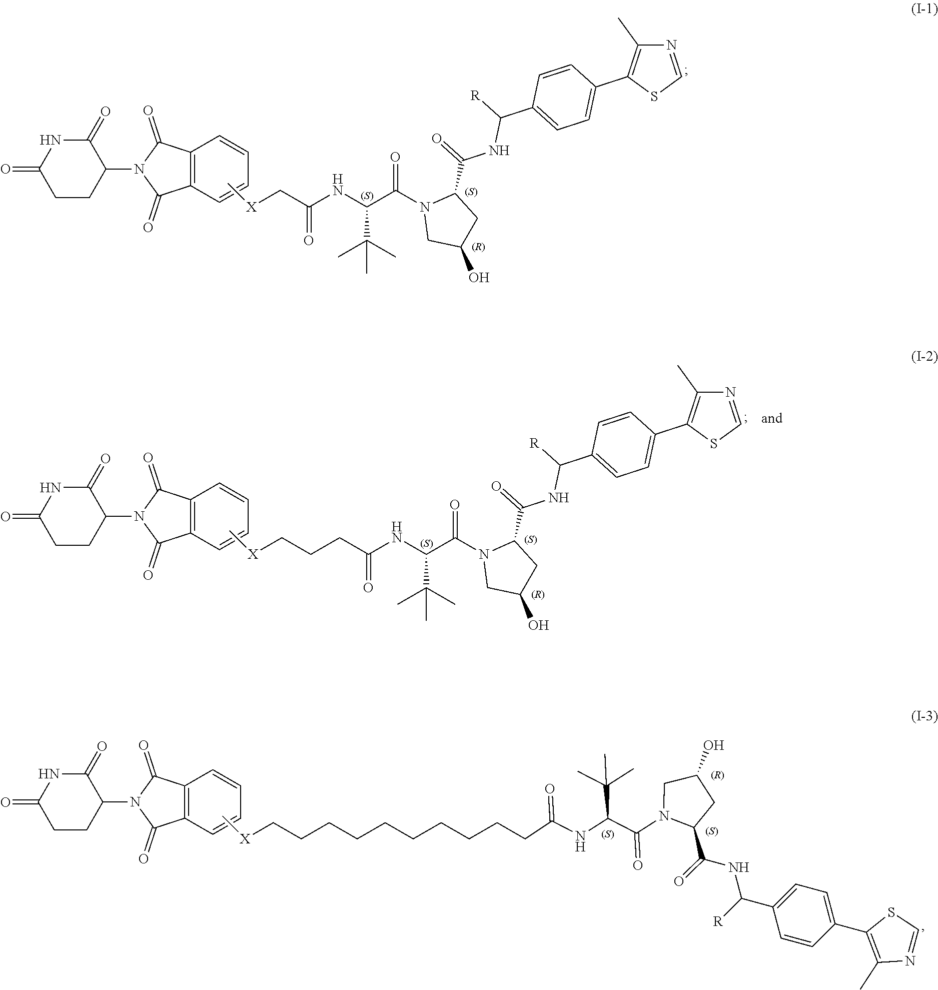
or a pharmaceutically acceptable salt or stereoisomer thereof.

6. The bispecific compound of claim 1, which is represented by any one of structures 1-3, 5, and 6: 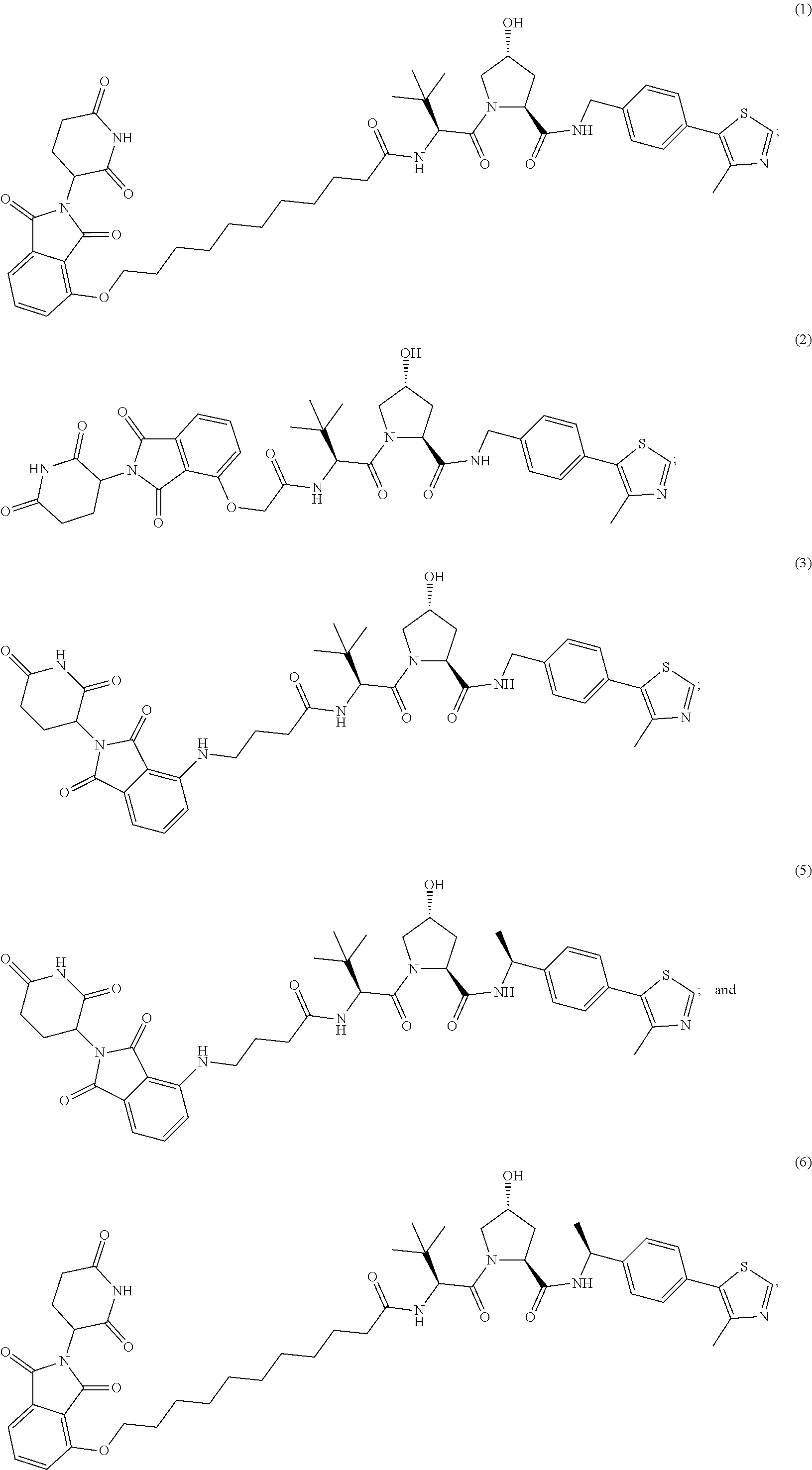
or a pharmaceutically acceptable salt and stereoisomer thereof.

7. A pharmaceutical composition, comprising a therapeutically effective amount of the bispecific compound of claim 1, or a pharmaceutically acceptable salt or stereoisomer thereof, and a pharmaceutically acceptable carrier.

8. A method of reducing an adverse side effect associated with a cereblon (CRBN) targeted therapy, comprising administering to a patient in need thereof a therapeutically effective amount of the bispecific compound of claim 1 or pharmaceutically acceptable salt or stereoisomer thereof.

9. A method of using the bispecific compound of claim 1 as a tool for studying CRBN biology via chemically induced knockdown.

10. The method of claim 9, which is conducted in vitro.

11. The method of claim 9, which is conducted in vivo in a non-human animal.

12. The method of claim 9, wherein the non-human animal is a rodent.

13. The bispecific compound of claim 1, which is: 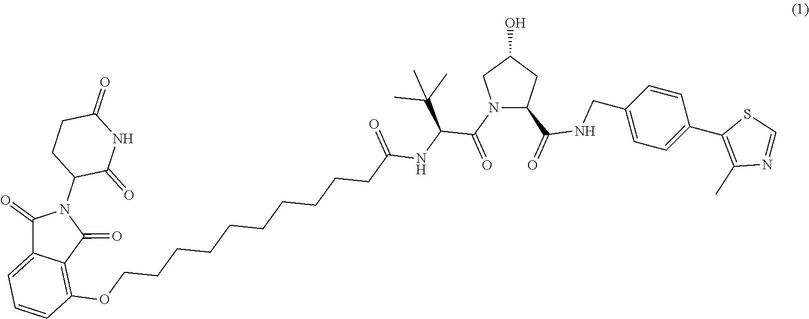 [SMILES: Cc1ncsc1-c1ccc(CNC(=O)[C@@H]2C[C@@H](O)CN2C(=O)[C@@H](NC(=O)CCCCCCCCCCOc2cccc3c2C(=O)N(C2CCC(=O)NC2=O)C3=O)C(C)(C)C)cc1]
or a pharmaceutically acceptable salt and stereoisomer thereof.

14. The bispecific compound of claim 1, which is: 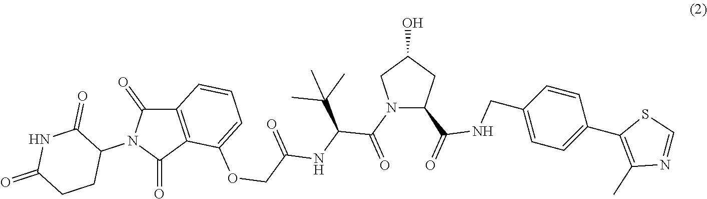 [SMILES: Cc1ncsc1-c1ccc(CNC(=O)[C@@H]2C[C@@H](O)CN2C(=O)[C@@H](NC(=O)COc2cccc3c2C(=O)N(C2CCC(=O)NC2=O)C3=O)C(C)(C)C)cc1]
or a pharmaceutically acceptable salt and stereoisomer thereof.

15. The bispecific compound of claim 1, which is: 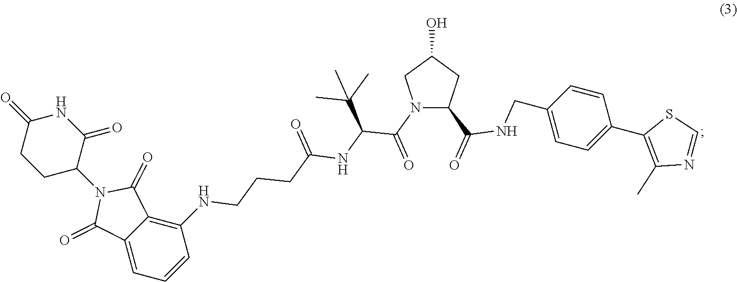 [SMILES: Cc1ncsc1-c1ccc(CNC(=O)[C@@H]2C[C@@H](O)CN2C(=O)[C@@H](NC(=O)CCCNc2cccc3c2C(=O)N(C2CCC(=O)NC2=O)C3=O)C(C)(C)C)cc1]
or a pharmaceutically acceptable salt and stereoisomer thereof.

16. The bispecific compound of claim 1, which is: 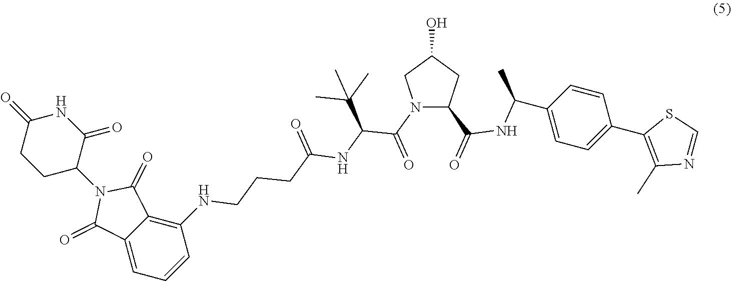 [SMILES: Cc1ncsc1-c1ccc([C@H](C)NC(=O)[C@@H]2C[C@@H](O)CN2C(=O)[C@@H](NC(=O)CCCNc2cccc3c2C(=O)N(C2CCC(=O)NC2=O)C3=O)C(C)(C)C)cc1]
or a pharmaceutically acceptable salt and stereoisomer thereof.

17. The bispecific compound of claim 1, which is: 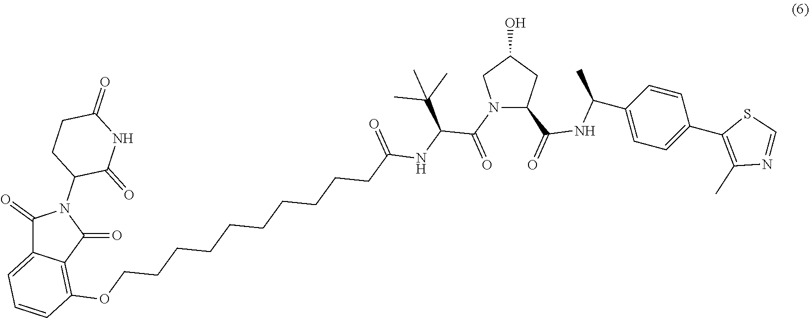 [SMILES: Cc1ncsc1-c1ccc([C@H](C)NC(=O)[C@@H]2C[C@@H](O)CN2C(=O)[C@@H](NC(=O)CCCCCCCCCCOc2cccc3c2C(=O)N(C2CCC(=O)NC2=O)C3=O)C(C)(C)C)cc1]
or a pharmaceutically acceptable salt and stereoisomer thereof.

18. A pharmaceutical composition, comprising a therapeutically effective amount of the bispecific compound of claim 6 or a pharmaceutically acceptable salt or stereoisomer thereof, and a pharmaceutically acceptable carrier.

19. A method of reducing an adverse side effect associated with a CRBN targeted therapy, comprising administering to a patient in need thereof a therapeutically effective amount of the bispecific compound of claim 6 or pharmaceutically acceptable salt or stereoisomer thereof.

20. A method of using the bispecific compound of claim 6 as a tool for studying CRBN biology via chemically induced knockdown.
